(19) United States
(12) Patent Application Publication
(10) Pub. No.: US 2007/0106274 A1
(43) Pub. Date: May 10, 2007

(54) Control systems for implantable medical devices

(76) Inventors: Peter Joseph Ayre, Crows Nest (AU); Lee Thomas Glanzmann, Darlington (AU); Nicholas Oliver Von Huben, Bexley North (AU)

Correspondence Address: LADAS & PARRY LLP, 224 SOUTH MICHIGAN AVENUE, SUITE 1600, CHICAGO, IL, 60604, US

(21) Appl. No.: 11/581,615

(22) Filed: Oct. 16, 2006

(30) Foreign Application Priority Data
Oct. 19, 2005 (AU) 2005 905782

(51) Int. Cl. A61K 9/22; A61M 5/32

(52) U.S. Cl. 604/890.1; 604/174

(57) ABSTRACT

A control system for use with an implantable medical device. Said control system includes a control circuit carried by a skin patch that is adapted to be attached to the outer surface of the skin layer of a patient.

### FIELD OF THE INVENTION

The present invention relates to improvements to control systems for implantable medical devices.

### BACKGROUND OF THE INVENTION

In the past, there have been many implantable medical devices that require bulky and sophisticated control systems. These control systems are generally mounted within a solid external housing or other mounting box. This housing or box is generally bulky and cumbersome to carry and use, particularly so for elderly or sick patients, and if they are to be carried for an extended period of time. Some implantable medical devices may require the patient to carry around the control system and/or the medical device for the rest of their life. These sorts of implantable medical devices may include implantable blood pumps or left ventricular assist devices.

U.S. Pat. No. 6,895,280—Meadows et al, discloses a control system for an implantable medical device wherein the control circuit is mounted in a relatively bulky and rigid box type housing. U.S. Pat. No. 6,889,084—Thompson et al, discloses a control system that includes a miniaturized control circuit with an RF transmission system. Even though this control system and circuit has been miniaturized, the control system may be considered to be relatively bulky as the control system is housed within a box that is not mounted on the skin of the patient.

Therefore, there has been a long-felt need for a control system for use with an implantable medical device that minimises the bulk and addresses patient's physical needs. There has also been a long-felt need to improve patient comfort. Many previous devices and systems are not designed with consideration to the needs of patients using medical devices for extended periods of time.

The present invention aims to, or at least address or ameliorate, one or more of the disadvantages associated with the above-mentioned prior art.

### SUMMARY OF THE INVENTION

In accordance with a first aspect the present invention consists of a control system for use with an implantable medical device, wherein said control system includes a control circuit carried by a skin patch that is adapted to be attached to the outer surface of the skin layer of a patient.

Preferably, said control circuit is encapsulated within said skin patch.

Preferably, said skin patch is made from a mouldable material.

Preferably, said mouldable material is silicone.

Preferably, said patch is bendable or deformable to suit anatomical positioning.

Preferably, said control circuit includes a vibrating alarm.

Preferably, the outer surface of said patch is partially coated with adhesive.

Preferably, said implantable medical device is an implantable rotary blood pump.

In accordance with a second aspect the present invention consists of a skin patch adapted to be attached to the outer surface of the skin layer of a patient, said patch carrying a control circuit for use with an implantable medical device.

Preferably, said skin patch is made from a mouldable material.

Preferably, said control circuit is encapsulated within said mouldable material.

Preferably, said patch is bendable or deformable to suit anatomical positioning.

Preferably, said control circuit includes a vibrating alarm.

Preferably, the outer surface of said patch is partially coated with adhesive.

In accordance with a third aspect the present invention consists of a control system for use with an implantable blood pump, said system comprising a control circuit carried by a bendable skin patch adapted to be attached to the outer skin layer of a patient.

Preferably, said control circuit includes a vibrating alarm.

### BRIEF DESCRIPTION OF DRAWINGS

The embodiments of the present invention will now be described with reference to the accompanied drawings wherein:

FIG. 1 depicts a top view of a first embodiment of the present invention;

FIG. 2 depicts a side cross-sectional view of the first embodiment as depicted in FIG. 1.

FIG. 3 depicts a perspective view of a control circuit in accordance with a second embodiment of the present invention.

### BRIEF DESCRIPTION OF PREFERRED EMBODIMENTS

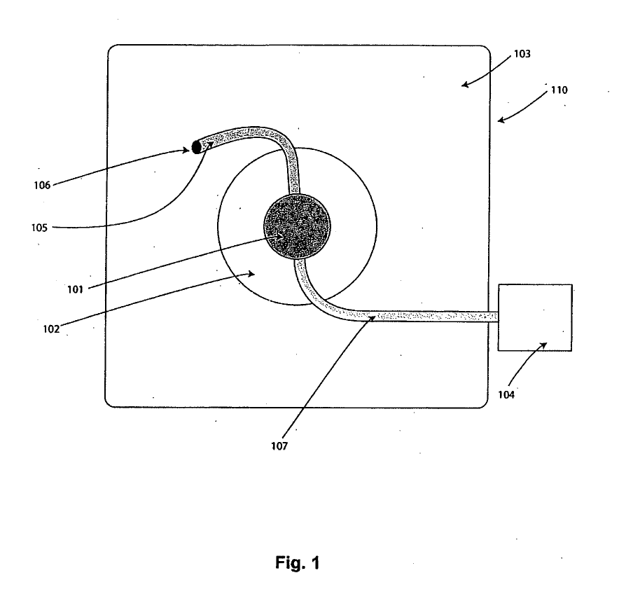

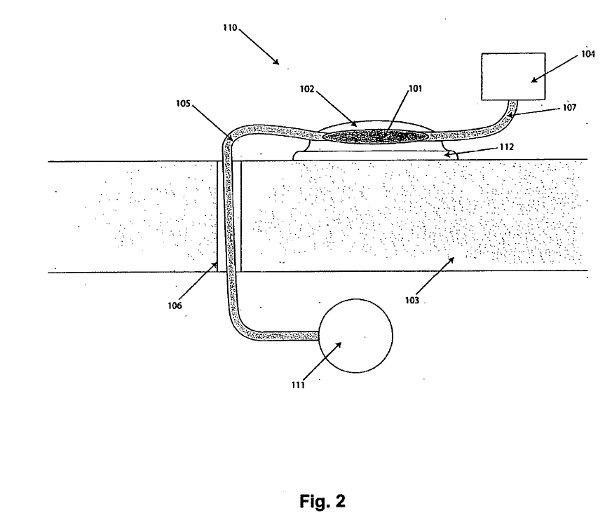

In FIGS. 1 and 2, a first embodiment of the present invention is depicted and includes a control system 110. The control system 110 includes a control circuit 101 encapsulated within a patch 102 mounted on the skin layer 103 of a patient.

Preferably, the control circuit 101 is electrically connected to a power source 104 via a first set of wires 107. The control circuit 101 is also electrically connected to an implantable medical device 111, which in this embodiment is a left ventricle assist device, via a second set of wires 105. This left ventricular assist device 111 may be an implantable rotary blood pump such as the VentrAssist™ as described in U.S. Pat. No. 6,227,797—Watterson et al. This particular US patent is included within the description of the present invention forms part of this description.

In this arrangement, the control circuit 101 regulates, provides power to, and extracts data from, the left ventricle assist device 111. Power source 104 provides electrical power to power circuit 101 which then supplies electrical power onto the implantable medical device 111.

The power source 104 may be replaced with either: a mains power connection; a single battery; or a combination thereof. Please note that this power source 104 may be adapted to be suitable for various implantable medical device 111.

Preferably, the second set of wires 105 may be in the form of a percutaneous lead which exits the skin layer 103 of a patient through a permanent exit wound 105.

Preferably, control circuit 101 may be encapsulated within a patch 102. The patch 102 may be generally flexible (or bendable) so the patch may be twisted, bent or otherwise deformed so as to adapt to the region of the outer skin layer 103 upon which it is mounted. Preferably, the patch 102 includes either a biocompatible or external use medical adhesive which is capable of adhering said patch 102 to the outer skin layer 103 of a patient. The patch 102 may be moulded from a materials including silicone. Preferably, the adhesive 112 is suitably strong enough to resist pulling or tearing associated with accidental movement of the first set of wires 107 or the power source 104.

The control circuit 101 may also preferably include a control system as disclosed in U.S. Pat. No. 6,866,625—Ayre et al. The description of this patent is included within this specification herein.

The control circuit 101, being encapsulated within patch 102, may allow the entire control arrangement 110 to be overall less bulky than comparable systems. The reduction of bulk may lead to increases in patient comfort. The reduction of weight may also lead to reduced incidence of accidental wear and tear on the percutaneous lead assembly formed by the combination of the first set of wires 107 and the second set of wires 105.

The portion of the percutaneous lead assembly exiting through skin layer 103 may be replaced with a Transcutaneous Energy Transmission System (“TETS”) and eliminating the need for a permanent exit wound 106 in the skin layer 103 of the patient. TETS is a system whereby energy, power, and data may be transmitted transcutaneously across the skin layer 103 by use of RF transmission to the implantable medical device 111 using electromagnetic coils (not shown) mounted on either side of the skin layer of the skin to provide a means for power transmission by RF frequencies.

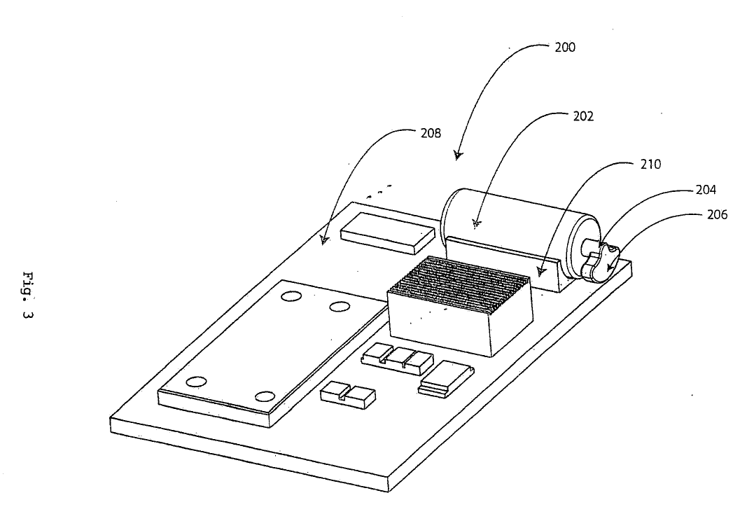

In a second embodiment the control system of the present invention may comprise a control circuit 200 on a printed circuit board 208 that includes a specialised alarm, as shown in FIG. 3. The control circuit 200 is encapsulated in a patch (not shown) that is similar to patch 102 of the first embodiment, and would be used for an implantable medical device 111 similar to that referred to earlier. The specialised alarm may be in the form of a miniature motor 202 mounted on or in the control circuit 200 via a support bracket 210. The rotor 204 of the miniature motor 202 is connected to an extrinsic arm 206, that is not balanced around the centre of its rotation, such that rotation of the arm 206 causes a small vibration. This vibration, when the motor 202 is mounted to the control circuit 200, is sufficient to cause a vibration of the control circuit 200 and the patch. This vibration may be then passed to the skin layer of the patient wherein the patient will feel a small vibration and be alerted, when necessary. This alarm may be a significant improvement over previous audible or visual alarms as it will alert the patient to a problem raised by the control circuit 200 no matter what the physical conditions of the patient are including, but not limited to, high levels of background noise or high levels of background light, which would otherwise make other types of alarms inappropriate.

Whilst the implantable medical device 111 referred to in the abovementioned first embodiment is a left ventricle assist device, it should be understood that the present invention may be used with other types of implantable medical devices.

The above descriptions detail only some of the embodiments of the present invention. Modifications may be obvious to those skilled in the art and may be made without departing from the spirit and the scope of the present invention.

1. A control system for use with an implantable medical device, wherein said control system includes a control circuit carried by a skin patch that is adapted to be attached to the outer surface of the skin layer of a patient.

2. A control system as claimed in claim 1, wherein said control circuit is encapsulated within said skin patch.

3. A control system as claimed in claim 1, wherein said skin patch is made from a mouldable material.

4. A control system as claimed in claim 3, wherein said mouldable material is silicone.

5. A control system as claimed in claim 1, wherein said patch is bendable or deformable to suit anatomical positioning.

6. The control system as claimed in claim 1, wherein said control circuit includes a vibrating alarm.

7. The control system as claimed in claim 1, wherein the outer surface of said patch is partially coated with adhesive.

8. The control system as claimed in claim 1, wherein said implantable medical device is an implantable rotary blood pump.

9. A skin patch adapted to be attached to the outer surface of the skin layer of a patient, said patch carrying a control circuit for use with an implantable medical device.

10. A skin patch as claimed in claim 9, wherein said skin patch is made from a mouldable material.

11. A skin patch as defined in paragraph 9, wherein said control circuit is encapsulated within said mouldable material.

12. A skin patch as claimed in claim 9, wherein said patch is bendable or deformable to suit anatomical positioning

13. A skin patch as claimed in claim 9, wherein said control circuit includes a vibrating alarm.

14. A skin patch as claimed in claim 9, wherein the outer surface of said patch is partially coated with adhesive.

15. A control system for use with an implantable blood pump, said system comprising a control circuit carried by a bendable skin patch adapted to be attached to the outer skin layer of a patient.

16. A control system as claimed in Claim 15, wherein said control circuit includes a vibrating alarm.
